(19) United States
(12) Patent Application Publication
(10) Pub. No.: US 2025/0204851 A1
(43) Pub. Date: Jun. 26, 2025

(54) IMAGING APPARATUS AND METHOD FOR DETECTING BLOOD VESSELS UNDER SKIN USING TRANSMISSIVE LIGHT SOURCES

(71) Applicant: IISM INC., Sejong (KR)

(72) Inventors: Il Seung Yang, Sejong (KR); Yun Mi Bai, Sejong (KR); Yu Sic Kim, Sejong (KR); Kwan Kyu Lee, Sejong (KR); Youn Jin Kim, Daejeon (KR); Chang Hyun Bae, Sejong (KR)

(21) Appl. No.: 18/847,641

(22) PCT Filed: Mar. 13, 2023
(86) PCT No.: PCT/KR2023/003349

§ 371 Date: Sep. 16, 2024

(30) Foreign Application Priority Data
Apr. 13, 2022 (KR) 10-2022-0045560

(51) Int. Cl. A61B 5/00

(52) U.S. Cl. CPC A61B 5/489; A61B 5/0077

(57) ABSTRACT

An apparatus and method for detecting blood vessels under the skin using transmissive light sources are provided. The apparatus for detecting blood vessels under the skin according to one embodiment of the present application comprises a light source unit, a fix unit, an image processing unit, and an image display unit, wherein the light source unit is located on a flexible plane and includes one or more infrared light sources; the fix unit is located at an end of the flexible plane; the infrared light sources are disposed to follow a surface of the subject as the flexible plane surrounds the subject and the flexible plane is fixed by means of the fix unit; a part of light irradiated toward the subject from the disposed infrared light sources is reflected from the subject, and another part of the light is transmitted through the subject; the image processing unit comprises optical lens for receiving the light reflected from the subject and the light transmitted through the subject, an infrared transmitting filter for transmitting the light passing through the optical lens, and a camera detection unit for detecting the light transmitted through the infrared transmitting filter; and the image display unit outputs image information detected by means of the camera detection unit.

### RELATED APPLICATIONS

This application is a 371 National Stage of International Patent Application No. PCT/KR2023/003349 filed Mar. 13, 2023, entitled, which claims priority to Korean Patent Application No. 10-2022-0045560 filed on Apr. 13, 2022, the disclosure of which each is incorporated herein in their entireties by reference.

### TECHNICAL FIELD

The present invention relates to a system for detecting blood vessels under the skin, which is capable of visualizing blood vessels located under the skin using infrared light sources, and more specifically, to a system for detecting blood vessels under the skin, which can obtain a high-quality image by combining transmissive infrared light sources and reflective infrared light sources.

### BACKGROUND ART

In the medical and cosmetic industries, it is very important to accurately detect the location of blood vessels under the skin that are difficult to observe, in order to prevent erroneous surgery or procedure.

An apparatus for detecting blood vessels under the skin using transmissive infrared light sources is disclosed in literatures such as KR 10-1999-0083994 A. However, such apparatus has a disadvantage in that only two-dimensional blood vessel detection is possible, and three-dimensional blood vessel detection is difficult. In addition, there is a drawback in that it is difficult to identify blood vessels under the skin that are difficult to observe because the depth at which infrared rays penetrate is limited.

Furthermore, in the case of the prior art technology for detecting blood vessels under the skin using visible light rather than infrared light have, there are drawbacks in that light (visible light) irradiated for detecting blood vessels under the skin causes pain in the eye and high-quality blood vessel images cannot be obtained.

### DETAILED DESCRIPTION OF THE INVENTION

### Technical Task

To address the aforementioned drawbacks of the prior art, an object of the present invention is to provide an image and method for detecting blood vessels under the skin, which allow infrared rays to penetrate deeper under the skin, enable 3D blood vessel detection, and obtain high-quality images without causing pain in the eye.

In addition, another object of the present invention is to provide an image and method for detecting blood vessels under the skin, which reduce noise.

The problems addressed by the present disclosure are not limited thereto, and may be variously expanded without departing from the idea and scope of the present invention.

### Means for Achieving the Task

According to one embodiment of the present invention to achieve the objects of the present invention, an apparatus for detecting blood vessels under the skin comprises a light source unit, a fix unit, an image processing unit, and an image display unit, wherein the light source unit is located on a flexible plane and includes one or more infrared light sources; the fix unit is located at an end of the flexible plane; the infrared light sources are disposed to follow a surface of the subject as the flexible plane surrounds the subject and the flexible plane is fixed by means of the fix unit; a part of light irradiated toward the subject from the disposed infrared light sources is reflected from the subject, and another part of the light is transmitted through the subject; the image processing unit comprises optical lens for receiving the light reflected from the subject and the light transmitted through the subject, an infrared transmitting filter for transmitting the light passing through the optical lens, and a camera detection unit for detecting the light transmitted through the infrared transmitting filter; and the image display unit outputs image information detected by means of the camera detection unit.

According to another embodiment of the present invention, the apparatus for detecting blood vessels under the skin may further comprise an observation unit that is located on the flexible plane and allows the subject to be observed.

According to another embodiment of the present invention, the apparatus for detecting blood vessels under the skin may further comprise a control unit that is located on the flexible plane and configured to control the intensity of the infrared light sources.

In the apparatus for detecting blood vessels under the skin according to another embodiment of the present invention, the distance or angle of the infrared light sources with respect to the subject may be controlled by means of the fix unit.

In the apparatus for detecting blood vessels under according to another embodiment of the present invention, the infrared light sources may be disposed on the light source unit at regular intervals.

According to another one embodiment of the present invention to achieve the objects of the present invention, a method for detecting blood vessels under the skin comprises the steps of: disposing infrared light sources included in a light source unit such that the infrared light sources follow a surface of a subject, wherein the light source unit is located on a flexible plane and includes one or more infrared light sources, and the fix unit is located at an end of the flexible plane, and the infrared light sources are disposed to follow the surface of the subject as the flexible plane surrounds the subject and the flexible plane is fixed by means of the fix unit; irradiating an infrared ray to the subject by means of the light source unit, wherein a part of light irradiated toward the subject from the disposed infrared light sources is reflected from the subject, and another part of the light is transmitted through the subject; passing the light reflected from the subject and the light transmitted through the subject through an infrared transmitting filter; detecting the light passed through the infrared transmitting filter by means of a camera detection unit; and displaying image information detected by the camera detection unit on an image display unit.

According to another one embodiment of the present invention, the method for detecting blood vessels under the skin may further comprise the step of observing the subject by means of the observation unit located on the flexible plane.

According to another one embodiment of the present invention, the method for detecting blood vessels under the skin may further comprise the step of controlling the intensity of the infrared light sources by means of a control unit located on the flexible plane.

According to another one embodiment of the present invention, the method for detecting blood vessels under the skin may further comprise the step of controlling the distance or angle of the infrared light sources with respect to the subject by means of the fix unit.

According to the method for detecting blood vessels under the skin according to another embodiment of the present invention, the infrared light sources may be disposed on the light source unit at regular intervals.

### Effect of the Invention

An apparatus and method for detecting blood vessels under the skin according to the present invention are configured such that the infrared light sources are disposed to follow a surface of a subject, and thus not only the light reflected from the subject but also the light transmitted through the subject are used for detecting blood vessels under the skin, thereby allowing infrared rays to penetrate deeper under the skin and enabling three-dimensional detection for blood vessels under the skin.

In addition, the apparatus and method for detecting blood vessels under the skin according to the present invention comprise the fix unit and the control unit, thereby controlling the distance, angle, or intensity of the infrared light sources with respect to the subject, and thus obtaining high-quality blood vessel images.

Moreover, the apparatus and method for detecting blood vessels under the skin according to the present invention use transmissive light sources so as to prevent diffuse reflection on the surface of the skin, which may occur in the case of the prior art using only reflected infrared rays, thereby preventing or remarkably reducing noise.

Furthermore, the apparatus and method for detecting blood vessels under the skin according to the present invention enables the detection for blood vessels under the skin for various body parts by positioning the light source unit and the fix unit on the flexible plane.

### BRIEF DESCRIPTION OF THE DRAWINGS

FIG. 1 illustrates an apparatus for detecting blood vessels under the skin according to one embodiment of the present invention.

FIG. 2 illustrates a flexible plane of the apparatus for detecting blood vessels under the skin according to one embodiment of the present invention.

FIG. 3 illustrates a system diagram of the apparatus for detecting blood vessels under the skin according to one embodiment of the present invention.

FIG. 4(a) illustrates the principle for detecting blood vessels under the skin in the conventional apparatus for detecting blood vessels under the skin.

FIG. 4(b) illustrates the principle for detecting blood vessels under the skin in the apparatus for detecting blood vessels under the skin according to the present invention.

FIG. 5(a) illustrates an image for detecting blood vessels under the skin obtained by the conventional principle for detecting blood vessels under the skin.

FIG. 5(b) illustrates an image for detecting blood vessels under the skin obtained by the principle for detecting blood vessels under the skin according to the present invention.

FIG. 6 illustrates one embodiment using the apparatus for detecting blood vessels under the skin according to the present invention.

### DETAILED DESCRIPTION FOR CARRYING OUT THE INVENTION

Hereinafter, the preferred embodiments of the present invention will be described in detail with reference to the accompanying drawings. Advantages and features of the present invention and methods for achieving the same will become apparent with reference to the embodiments described in detail below together with the accompanying drawings. However, the present invention is not limited to the embodiments described below, but may be implemented in various different manners, and the embodiments of the present invention are provided so as to complete the disclosure of the present invention and to completely inform a person having ordinary skill in the art to which the present invention pertains of the scope of the present invention, and the present invention is defined only by the scope of the claims. The same reference numerals refer to the same components throughout the specification.

Unless otherwise defined, all terms (including technical and scientific terms) used in the present specification have the same meaning as commonly understood by a person having ordinary skill in the art to which the present invention pertains. In addition, terms defined in commonly used dictionaries are not interpreted ideally or excessively unless they are clearly specifically defined.

The terms used in the present specification are to explain the embodiments and are not intended to limit the present invention. In the specification, a singular term includes a plural term unless specifically mentioned in the text. The term ‘comprise’ and/or ‘comprising’ used in the specification does not exclude the presence or addition of one or more other components in addition to the mentioned components.

Hereinafter, the apparatus and method for detecting blood vessels under the skin according to the embodiments of the present invention will be described with reference to the drawings.

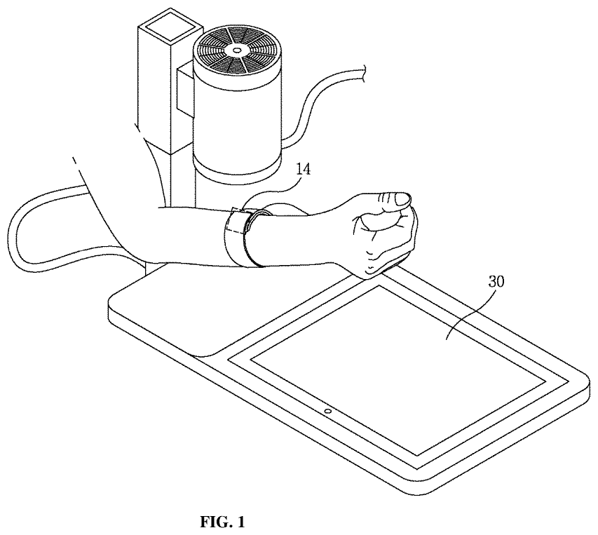

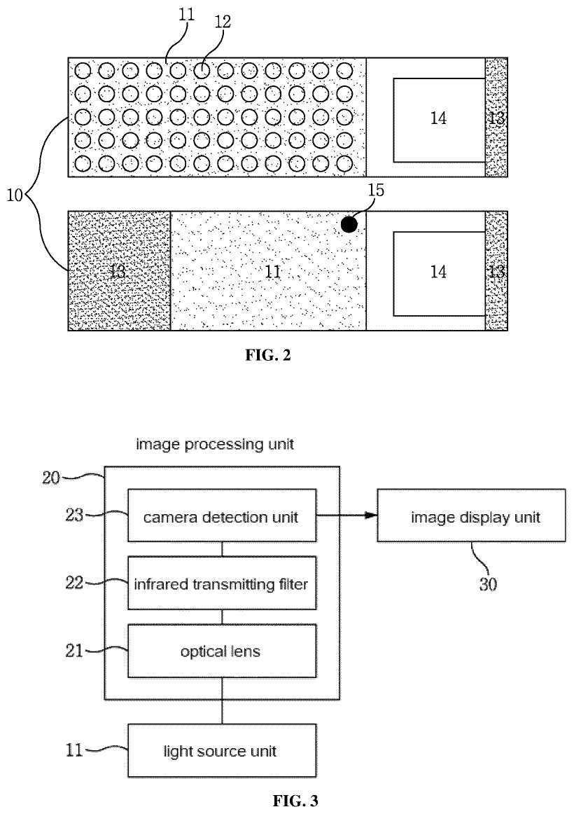

FIG. 1 illustrates an apparatus for detecting blood vessels under the skin according to one embodiment of the present invention; FIG. 2 illustrates a flexible plane of the apparatus for detecting blood vessels under the skin according to one embodiment of the present invention; and FIG. 3 illustrates a system diagram of the apparatus for detecting blood vessels under the skin according to one embodiment of the present invention.

The apparatus for detecting blood vessels under the skin according to one embodiment of the present invention comprises a light source unit 11, a fix unit 13, an image processing unit 20, and an image display unit 30. Specifically, the light source unit 11 and the fix unit 13 will be explained, and then the image processing unit 20 and the image display unit 30 will be explained.

The light source unit 11 is located on a flexible plane 10 and includes one or more infrared light sources 12. The infrared light sources 12 may be an infrared LED, an incandescent bulb, a halogen, a sodium lamp, or an EL (electromagnetic) lamp, but may be any form as long as it can emit infrared light, but is not limited thereto. The one or more infrared light sources 12 may be disposed on the light source unit 11 in various forms. For example, the infrared light sources 12 may be disposed on the light source unit 11 at regular intervals.

The fix unit 13 is located on the flexible plane 10. The fix unit 13 may be located at any point on the flexible plane 10, preferably at an end of the flexible plane 10. In addition, the fix unit 13 may be located on one surface, the other surface, or both surfaces of the flexible plane 10. The fix unit 13 may be made of any material as long as it can be bonded between the fix units 13, and may be, for example, a Velcro, a snap button, a button, or the like.

The flexible plane 10 is a surface that is flexible in nature and may be bent by pressure, and the thickness, material, and the like are not limited. The flexible plane 10 may be bent by applying the pressure to the flexible plane 10, and the flexible plane 10 may be rolled up by the bonding between the fix units 13. For example, the flexible plane 10 may be bent while surrounding the subject, and the flexible plane 10 may be maintained or fixed in a state where the flexible plane 10 surrounds the subject by means of the fix unit 13. The width of the fix unit 13 on the flexible plane 10 may be freely controlled so that the distance or angle of the infrared light sources 12 with respect to the subject may be freely controlled. Furthermore, when the flexible plane 10 is rolled up, its diameter may also be freely controlled. Through this configuration, the apparatus for detecting blood vessels under the skin according to the present invention can be disposed or fixed on various body parts, thereby enabling the detection for blood vessels under the skin for various body parts.

The flexible plane 10 may be rolled up so that, out of the both surface, the surface on which the light source unit 11 is located is directed toward the subject, and the infrared light sources 12 of the light source unit 11 may be disposed to follow the surface of the subject. The infrared light sources 12 disposed to follow the surface of the subject may irradiate infrared rays toward the subject at various angles. When infrared rays are irradiated to the subject through the infrared light sources 12 disposed as described above, the light may be reflected from the subject or transmitted through the subject. The apparatus for detecting blood vessels under the skin according to the present invention uses the light reflected from the subject and/or the light transmitted through the subject, which allows infrared rays to penetrate deeper under the skin, enables 3D blood vessel detection, and provide high-quality images without causing pain in the eye. In addition, the apparatus for detecting blood vessels under the skin according to the present invention irradiates the light to the subject at various angles, thereby preventing diffuse reflection on the surface of the skin, which may occur in the case of the prior art using only reflected infrared rays, thereby preventing or remarkably reducing noise.

The image processing unit 20 comprises an optical lens 21 for receiving the light reflected from the subject and the light transmitted through the subject, an infrared transmitting filter 22 for transmitting the light passing through the optical lens 21, and a camera detection unit 23 for detecting the light transmitted through the infrared transmitting filter 22. The image processing unit 20 may be a device capable of processing image information obtained with its own central processing unit (CPU), and may be, for example, a personal computer (PC) of a Windows operating system, a Linux-based PC, an Android-based tablet PC, or the like, but is not limited thereto.

The apparatus for detecting blood vessels under the skin according to the present invention enables the detection of blood vessels under the skin in the manner of irradiating invisible infrared rays toward the subject using infrared light sources, and observing by means of the camera the light reflected from the subject and/or the light transmitted through the subject.

More specifically, the apparatus for detecting blood vessels under the skin according to the present invention may receive the light reflected from the subject and/or the light transmitted through the subject, which are irradiated from the infrared light sources 12 of the light source unit 11, by means of the optical lens 21. In addition, the optical lens of the present invention may be optical zoom lens, and by using the optical zoom lens, an optically enlarged image can be obtained. Unlike screen enlargement through digital image processing, by enlarging the original image itself using a lens, there is no blurring of the image, and a clear enlarged image can be obtained. Based on this, it is possible to clearly detect capillaries that are thin so cannot be identified. In addition, a shutter may be mounted inside the optical lens to include the function of controlling the intensity of an incoming image.

The apparatus for detecting blood vessels under the skin according to the present invention may effectively remove noise by passing the light, which is irradiated from the infrared light sources 12 of the light source unit 11 and reflected from or transmitted through the subject, through the infrared transmitting filter 22 before being detected by means of the camera detection unit 23. The infrared transmitting filter 22 is not used to selectively filter the wavelength of the light source, but is used to prevent noise caused by the external light source from entering the detector. For example, since the infrared ray LED has a narrow wavelength region of 10 nm, this simultaneously has the effect of a filter essentially required to select a specific wavelength when using a light source having a wideband wavelength such as a lamp; thus, this has the structure having a dual filtering effect as a whole. Since the absorption of blood by a specific wavelength is strengthened by the infrared LED, and noise by an external light source is blocked by the optical filter, the sharpness of the image may be maximized.

The apparatus for detecting blood vessels under the skin according to the present invention may obtain image information of blood vessels in the form of digital data by means of the camera detection unit 23, thereby easily processing the image information and visualizing the image information in a desired form. For example, since the digitized blood vessel image information is used, the exact location of the blood vessels may be visualized in various forms such as expressing the exact location of the blood vessels with a separate line, which may be helpful in detecting the accurate location of the blood vessels. In addition, various colors can be added to an image to realize the color and shape of an actual observation target so as to achieve the same effect as looking at a blood vessel which is actually present in the skin. Furthermore, the camera detection unit 23 may include one or more CCD (Charge-Coupled Device Camera) digital cameras or one or more CMOS (Complementary metal-oxide-semiconductor) cameras as an image sensor, and as the camera detection unit 23 includes the plurality of CCD digital cameras or CMOS cameras, an image of black-and-white blood vessels and an image of natural skin through can be obtained respectively, and the obtained images can be combined.

The image display unit 30 according to the present invention displays the image for detecting blood vessels under the skin processed by means of the image processing unit 20. In addition, the image display unit 30 may display a guide message indicating that the image for detecting blood vessels under the skin and/or the irradiation of light is correctly performed when the image obtained from the camera detection unit 23 satisfies a predetermined condition, and may display a warning or a guide phrase when the image obtained from the camera detection unit 23 does not satisfy the predetermined condition. Furthermore, the image display unit 30 may display various types of information required to use the apparatus for detecting blood vessels under the skin according to the present invention. The image display unit 30 of the present invention is an electronic device with a screen display to which a USB port can be connected, and may be, for example, a monitor, a beam projector, a digital picture frame, a TV, or the like, but is not limited thereto.

The apparatus for detecting blood vessels under the skin according to another embodiment of the present invention may further comprise an observation unit 14. The observation portion 14 may have a form in which a portion of the flexible plane 10 is open. As shown in FIG. 1, the observation portion 14 may be open in a square shape, or may be open in a circle, an ellipse, or a triangle shape. The shape of the observation portion 14 is not limited to the shape as described above, and may have various shapes as long as at least a portion of the flexible plane 10 is open. The apparatus for detecting blood vessels under the skin according to the present invention further comprises the observation unit 14, and the observation unit 14 has the opened form, thereby allowing the light to be irradiated to the subject, and at the same time, the subject to be observed, and a desired procedure, surgery, or blood collection to be performed on the subject. For example, when the flexible plane 10 of the present invention follows the wrist, the skin of the wrist may be observed by means of the observation portion 14, and a desired procedure, surgery, or blood collection can be performed on the skin of the wrist. In this case, the location of the blood vessels may be accurately identified by referring to the image for detecting blood vessels under the skin displayed on the image display unit 30, thereby enabling a desired procedure, surgery, or blood collection. Since a procedure, surgery, or blood collection may be performed by means of the observation unit 14 of the present invention, the observation unit 14 of the present invention may be referred to as an action unit.

The apparatus for detecting blood vessels under the skin according to another embodiment of the present invention may further comprise a control unit 15. The intensity of the infrared light sources 12 of the light source unit 11 may be controlled by means of the control unit 15 of the present invention.

Next, the operation principle for detecting blood vessels under the skin according to the present invention, and the method for detecting blood vessels under the skin will be explained.

The method for detecting blood vessels under the skin according to the present invention comprises the step of disposing infrared light sources 12 included in a light source unit 11 such that the infrared light sources 12 follow a surface of a subject. For example, the infrared light sources 12 of the light source unit 11 may be disposed to follow the surface of the subject as the flexible plane 10 surrounds the subject and the flexible plane 10 is fixed by means of the fix unit 13. In the present invention, the step of disposing infrared light sources 12 included in a light source unit 11 such that the infrared light sources 12 follow a surface of a subject is not limited to the aforementioned method, and it is only necessary that the infrared light sources 12 is disposed to follow the surface of the subject.

In addition, the method for detecting blood vessels under the skin according to the present invention comprises the step of irradiating an infrared ray to the subject by means of the light source 11. In the present invention, the step of irradiating an infrared ray to the subject may be performed after the step of disposing infrared light sources 12, and may be performed before the step of disposing infrared light sources 12. When the step of irradiating an infrared ray to the subject is performed after the step of disposing infrared light sources 12 is performed, a part of light irradiated toward the subject from the infrared light sources 12 is reflected from the subject and another part of the light irradiated toward the subject from the infrared light sources 12 is transmitted through the subject.

Below, the differences between the conventional method for detecting blood vessels under the skin and the method for detecting blood vessels under the skin according to the present invention, and the effects of the present invention resulting therefrom will be explained.

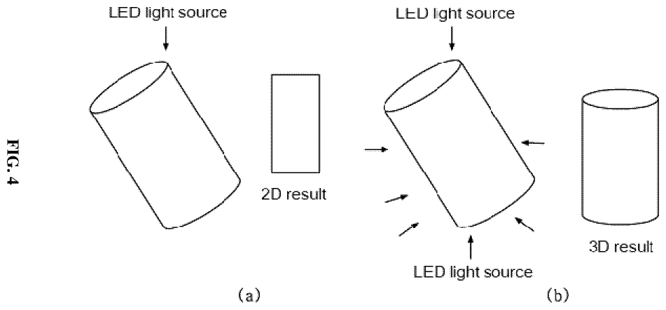

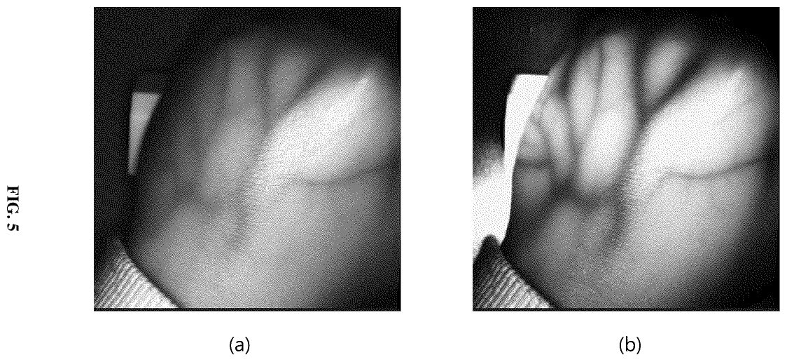

In the conventional method for detecting blood vessels under the skin, the blood vessels under the skin are detected by irradiating infrared rays to a subject and using light reflected from the subject. According to the conventional method for detecting blood vessels under the skin, as shown in FIG. 4A, cylindrical blood vessels under the skin can be observed only in a two-dimensional (2D) shape.

By contrast, in the method for detecting blood vessels under the skin according to the present invention, infrared light sources are disposed to follow the surface of the subject and infrared rays are irradiated to the subject. In this case, a part of light irradiated toward the subject from the infrared light sources is reflected from the subject, and another part of the light irradiated toward the subject from the infrared light sources is transmitted through the subject and is directed toward the optical lens 21 of the image processing unit 20.

According to the method for detecting blood vessels under the skin according to the present invention, as shown in FIG. 4B, the blood vessels under the skin may be observed in a three-dimensional (3D) shape, by using the light reflected from the subject and/or the light transmitted through the subject. The method for detecting blood vessels under the skin according to the present invention are configured such that the infrared light sources are disposed to follow the surface of a subject, and then, the light is irradiated to the subject, and the light reflected from the subject and/or the light transmitted through the subject is used, which allows infrared rays to penetrate deeper under the skin, enables three-dimensional detection for blood vessels under the skin, and provides high-quality images without causing pain in the eye. In addition, the method for detecting blood vessels under the skin according to the present invention irradiates the light to the subject at various angles, thereby preventing diffuse reflection on the surface of the skin, which may occur in the case of the prior art using only reflected infrared rays, thereby preventing or remarkably reducing noise.

More specifically, FIG. 5(a) illustrates a blood vessel detection result obtained according to the conventional method for detecting blood vessels under the skin using reflected infrared rays, and FIG. 5(b) illustrates a blood vessel detection result obtained according to the method for detecting blood vessels under the skin according to the present invention. As shown in FIG. 5, in the blood vessel detection result obtained according to the method for detecting blood vessels under the skin according to the present invention, the blood vessels were observed in a 3D shape, and the blood vessels under the skin that were difficult to observe according to the conventional method for detecting blood vessels under the skin were additionally observed.

According to the method for detecting blood vessels under the skin according to the present invention, light can be irradiated at various distances or angles, and the area in which the subject is exposed to the light source is increased, so that invisible blood vessels under the skin can be additionally observed. In addition, according to the conventional method for detecting blood vessels under the skin, it is difficult to visually identify blood vessels under the skin when the skin is thick, whereas according to the method for detecting blood vessels under the skin according to the present invention, blood vessels under the skin can be easily visually identified even when the skin is thick.

The method for detecting blood vessels under the skin according to the present invention comprises the step of passing the light reflected from the subject and/or the light transmitted through the subject through an infrared transmitting filter. Since the light passing through the optical lens 21 passes through the infrared transmitting filter 22 but other light cannot pass through, only the infrared image information including the image of blood vessels reaches the camera detection unit 23, thereby obtaining an effective noise removal effect.

The method for detecting blood vessels under the skin according to the present invention comprises obtaining the light passed through the infrared transmitting filter 22 by means of the camera detection unit 23 included in the image processing unit 20. Only the infrared image information including the image of the blood vessels passing through the infrared transmitting filter 22 reaches the camera detection unit 23.

The method for detecting blood vessels under the skin according to the present invention comprises the step of processing and generating image information using the light detected by means of the camera detection unit 23.

The method for detecting blood vessels under the skin according to the present invention comprises the step of outputting and displaying generated or processed image information. The step of outputting and displaying the image information may comprise a step of processing the image information, such as expressing the position of the blood vessels with a separate line or coloring the blood vessels so that the blood vessel information may be visualized in a desired shape by the operator, and outputting and displaying the image information.

The method for detecting blood vessels under the skin according to another embodiment of the present invention may further comprise a step of observing a subject by means of the observation unit 14 in which a part of a flexible plane is open.

The method for detecting blood vessels under the skin according to another embodiment of the present invention may further comprise a step of irradiating light to a subject and at the same time observing the subject by means of the observation unit 15, and performing a desired procedure, surgery, or blood collection on the subject. In this case, the location of the blood vessels may be accurately identified by referring to the image for detecting blood vessels under the skin displayed on the image display unit 30, thereby enabling a desired procedure, surgery, or blood collection.

The method for detecting blood vessels under the skin according to another embodiment of the present invention may further comprise a step of controlling the intensity of the infrared light sources 12 by means of a control unit 15 located on the flexible plane 10.

The method for detecting blood vessels under the skin according to another embodiment of the present invention may further comprise a step of controlling the distance or angle of the infrared light sources 12 with respect to the subject by means of the fix unit 13.

According to the method for detecting blood vessels under the skin according to another embodiment of the present invention, the infrared light sources 12 may be disposed on the light source unit 12 at regular intervals.

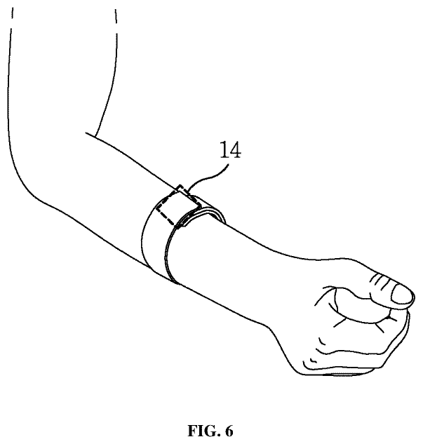

FIG. 6 illustrates one embodiment using the apparatus for detecting blood vessels under the skin according to the present invention. Specifically, this figure illustrates one embodiment in which the infrared light sources 12 of the light source 11 on the flexible plane 10 are disposed to follow the surface of the skin of the lower arm. The flexible plane 10 is fixed to surround the skin by the fix unit 13, and the observation portion 14 is disposed upward. In this case, according to the apparatus and method for detecting blood vessels under the skin according to the present invention, blood vessels under the skin of the lower arm can be accurately observed, and furthermore, a procedure, surgery, or blood collection for the lower arm can be performed by means of the observation unit (action unit).

The embodiments of the present invention are explained above with reference to the accompanying drawings, but it will be understood by those skilled in the art that the present invention can be implemented in other specific forms without changing the technical idea or essential feature. It should be noted, therefore, that the embodiments described above are exemplary and not limited in all aspects.

### EXPLANATIONS FOR REFERENCE NUMERALS

10: flexible plane
11: light source unit
12: infrared light source
13: fix unit
14: observation unit
15: control unit
20: image processing unit
21: optical lens
22: infrared transmitting filter
23: camera detection unit
30: image display unit

1. An apparatus for detecting blood vessels under the skin, comprising a light source unit, a fix unit, an image processing unit, and an image display unit, wherein
the light source unit is located on a flexible plane and includes one or more infrared light sources,
the fix unit is located at an end of the flexible plane,
the infrared light sources are disposed to follow a surface of the subject as the flexible plane surrounds the subject and the flexible plane is fixed by means of the fix unit,
a part of light irradiated toward the subject from the disposed infrared light sources is reflected from the subject, and another part of the light is transmitted through the subject,
wherein the image processing unit comprises:
optical lens for receiving the light reflected from the subject and the light transmitted through the subject;
an infrared transmitting filter for transmitting the light passing through the optical lens; and
a camera detection unit for detecting the light transmitted through the infrared transmitting filter, and
wherein the image display unit outputs image information detected by means of the camera detection unit.

2. The apparatus for detecting blood vessels under the skin according to claim 1, further comprising an observation unit form in which a portion of the flexible plane is open.

3. The apparatus for detecting blood vessels under the skin according to claim 1, further comprising a control unit that is located on the flexible plane and configured to control the intensity of the infrared light sources.

4. The apparatus for detecting blood vessels under the skin according to claim 1, wherein the distance or angle of the infrared light sources with respect to the subject is controlled by means of the fix unit.

5. The apparatus for detecting blood vessels under the skin according to claim 1, wherein the infrared light sources are disposed on the light source unit at regular intervals.

6. A method for detecting blood vessels under the skin comprises the steps of: disposing infrared light sources included in a light source unit such that the infrared light sources follow a surface of a subject, wherein the light source unit is located on a flexible plane and includes one or more infrared light sources, and the fix unit is located at an end of the flexible plane, and the infrared light sources are disposed to follow the surface of the subject as the flexible plane surrounds the subject and the flexible plane is fixed by means of the fix unit;
irradiating an infrared ray to the subject by means of the light source unit, wherein a part of light irradiated toward the subject from the disposed infrared light sources is reflected from the subject, and another part of the light is transmitted through the subject;
passing the light reflected from the subject and the light transmitted through the subject through an infrared transmitting filter;
detecting the light passed through the infrared transmitting filter by means of a camera detection unit; and
displaying image information detected by the camera detection unit on an image display unit.

7. The method for detecting blood vessels under the skin according to claim 6, further comprising: observing the subject by means of the observation unit in which a portion of the flexible plane is open.

8. The method for detecting blood vessels under the skin according to claim 6, further comprising: controlling the intensity of the infrared light sources by means of a control unit located on the flexible plane.

9. The method for detecting blood vessels under the skin according to claim 6, further comprising: controlling the distance or angle of the infrared light sources with respect to the subject by means of the fix unit.

10. The method for detecting blood vessels under the skin according to claim 6, further comprising: disposing the infrared light sources on the light source unit at regular intervals.
